(19) United States
(12) Patent Application Publication
(10) Pub. No.: US 2023/0215535 A1
(43) Pub. Date: Jul. 6, 2023

(54) METHOD FOR PROVIDING A MEDICAMENT COMBINATION AND DATA CARRIER WITH SOFTWARE

(71) Applicant: UNIVERSITÄT ULM, Ulm (DE)

(72) Inventor: Marc-Eric HALATSCH, Ulm (DE)

(73) Assignee: UNIVERSITÄT ULM, Ulm (DE)

(21) Appl. No.: 17/996,457

(22) PCT Filed: Apr. 20, 2021
(86) PCT No.: PCT/EP2021/060265

§ 371 Date: Oct. 18, 2022

(30) Foreign Application Priority Data
Apr. 22, 2020 (DE) 10 2020 205 076.7

(51) Int. Cl. G16H 20/10; G16H 50/20

(52) U.S. Cl. CPC G16H 20/10; G16H 50/20

(57) ABSTRACT

The invention relates to a method for providing a medication combination, wherein at least one medication of the medication combination to be provided is selected on the basis of the result of a molecular in vitro characterization of tumor tissue of a glioblastoma of a patient. The selection is made on the basis of eliminating and not selecting medications from a list of non-oncological medications for which no benefit has been acknowledged for treating the patient on the basis of the result of the molecular in vitro characterization. The medication combination which can be provided using the method is inexpensive, exhibits a low degree of toxicity, and is characterized by an improved efficacy in comparison to known medication combinations. The invention additionally relates to a data carrier with software which can support or simplify the process of carrying out the method according to the invention.

A method for providing a medicament combination is proposed with which a selection of at least one medicament of the medicament combination to be provided is made on the basis of the result of a molecular in-vitro characterization of tumour tissue of a glioblastoma of a patient. The basis for the selection is, in particular, that medicaments will be deleted from a list of non-oncological medicaments and not selected if, based on the result of the in-vitro molecular characterization, they are found to afford no benefit in the treatment of the patient. The medicament combination providable by the method is cost-effective, shows low toxicity and is characterized by improved effectiveness compared with known medicament combinations. The invention further provides a data carrier with software that can assist and simplify the execution of the method of the invention.

In the prior art, postoperative treatment of a glioblastoma of a patient is achieved through chemotherapy, radiation, further surgery and secondary—in the broadest sense of the term—drug therapies, which include reserve chemotherapies, immunological methods and “molecularly targeted therapies”.

A treatment approach that does not involve the use of biomarkers is known for this purpose (see e.g. Kast, E. et al., Oncotarget, volume 5, issue 18, pp. 8052-8082). This approach shows a promising effect in half of patients treated, but a lack of effectiveness in the other half, so there is potential for improvement here.

The use of personalized medicine in oncology, whereby medicaments are individually administered to a patient undergoing treatment according to particular molecular characteristics of the tumour tissue, is also known. However, in practice this strategy has not on its own proved a comprehensive success (see e.g. Le Tourneau, C. et al., Lancet Oncol., volume 16, issue 13, pages 1324-1334).

The disadvantages of the treatment approaches of the prior art are the limited therapeutic effectiveness of existing adjuvant drug treatment protocols. This is due to primary and secondary treatment resistance, in the latter case as a consequence of clonal selection and expansion of tumour cells during treatment. The outcome with current first-line therapy is a median overall survival from diagnosis of approx. 15 months. A considerable improvement in median overall survival is desirable.

Against this background, the object of the present invention was to specify a method with which a medicament combination can be provided that is cost-effective, has low toxicity and is characterized by improved effectiveness compared with known medicament combinations. In particular, the providable medicament combination (the medicament combination provided by the method) should be suitable for the postoperative treatment of glioblastoma of a patient. In addition, a data carrier with software should be provided that can assist and simplify the execution of the method.

The object is achieved by the method having the features of claim 1 and by the data carrier having the features of claim 10. The dependent claims illustrate advantageous embodiments.

The invention proposes a method for providing a medicament combination, comprising the steps:
a) performing a molecular in-vitro characterization of tumour tissue of a glioblastoma of a patient, with a result of the molecular in-vitro characterization being obtained;
b) providing a combination of medicaments, the combination comprising or consisting of an oncological medicament for the treatment of glioblastoma and at least nine non-oncological medicaments;
c) selecting at least one further medicament from a list of at least 100 non-oncological medicaments; and
d) combining the combination of medicaments provided in step b) with the at least one medicament identified in step c) into a medicament combination (i.e. drug combination);

characterized in that the selection of the at least one further medicament in step c) is based on the result of the molecular in-vitro characterization in step a), the basis for the selection being, in particular, that medicaments will be deleted from the list of at least 100 non-oncological medicaments and thus not selected if, based on the result of the in-vitro molecular characterization in step a), they are found to afford no benefit in the treatment of the patient.

The provided medicament combination is suitable in particular for the postoperative treatment of glioblastoma of a patient.

The expression “oncological medicament” means a medicament that at least in Germany had as of 20 Apr. 2020 marketing authorization for oncological treatment, in particular for the treatment of glioblastoma.

The expression “non-oncological medicament” means a medicament that at least in Germany had as of 20 Apr. 2020 marketing authorization not for the treatment of a tumour, in particular not for the treatment of glioblastoma. Where such medicaments are used for the treatment of a tumour, the expression “off-label use” is also used, with such medicaments also referred to in this context as “off-label” medicaments. Treatment of a tumour means treating a tumour directly, i.e. it does not mean symptomatic oncological treatment. For example, aprepitant is used as an antiemetic in chemotherapy and has marketing authorization for this. Aprepitant thus has marketing authorization in the context of symptomatic oncological treatment. However, aprepitant is a “non-oncological medicament” for the purposes of the present invention, since it did not at the abovementioned cutoff date have marketing authorization for the (direct) treatment of a tumour (per se).

The expression “no benefit in the treatment of a patient” means in particular that, based on the result of the molecular in-vitro characterization in step a) of the method according to the invention, the medicament is for the patient classified as not effective and/or not tolerated. Examples of effectiveness criteria are level of target molecule expression in the tumour, level of affinity between the drug and target molecule, strength of inhibition of a target molecule, level of lipophilicity of a drug and/or value of the model in which an off-label effect was demonstrated (cell-free system<cell culture<heterotopic in-vivo model<orthotopic in-vivo model<clinical case study<clinical case series<clinical phase I<II<Ill study). For the assessment of tolerability, drug interactions can be evaluated against a database (preferably with the aid of a software algorithm) based on the result for step a) in an iterative, permutative process in which various drug combinations are given, or will be given, an overall score. This allows the possibility of considering further only those drug combinations for which the drug interaction score conforms to a particular attribute (e.g. “minor” or “moderate” as opposed to “major”). These drug combinations are then used to select the most effective drug combination (preferably with the aid of a software algorithm). The algorithm may be stored on the data carrier of the invention and is in particular executed by the software stored on the data carrier of the invention.

The method of the invention allows a medicament combination to be provided that has greater effectiveness than currently used therapies in the postoperative treatment of glioblastoma of a patient. The greater effectiveness derives from the fact that the medicament combination includes not only an oncological medicament and at least nine non-oncological medicaments, but in addition at least one further non-oncological medicament specifically selected on the basis of the result of molecular in-vitro characterization of tumour tissue of a glioblastoma of a patient undergoing treatment. The medicament combination thus includes ten medicaments that were identified prior to execution of the method and at least one non-oncological medicament that was still unidentified prior to execution of the method and that was specifically selected for treating the patient undergoing treatment. The method is thus able to provide a medicament combination that arises from a combination of a biomarker-dependent and a biomarker-independent treatment approach.

It is plausible that the providable medicament combination is more effective than a medicament combination composed solely of the at least ten identified medicaments (i.e. the one oncological and the at least nine non-oncological medicaments) that serve as the fixed basis (“base cocktail”), since it is augmented by at least one non-oncological medicament (preferably by a plurality of non-oncological medicaments) (the “cocktail cap”). In addition, since the cocktail cap is tailored to the patient undergoing treatment, it constitutes a personalized medicament or personalized medicament combination. The plausibility of the improved effectiveness is moreover supported by the so-called “Nile Distributary Problem” (see Kast, E. et al., Oncotarget, volume 5, issue 18, pp. 8052-8082).

Furthermore, the medicament combination can be provided more cost-effectively, since a substantial part of it is composed of non-oncological medicaments (“off-label” medicaments).

Therapy with the providable medicament combination is thus more cost-effective than known adjuvant therapies.

In addition, the toxicity of the providable medicament combination is low, since it comprises primarily non-oncological medicaments.

The method can be characterized in that the molecular in-vitro characterization of tumour tissue of a glioblastoma of a patient is selected from the group consisting of in-vitro characterization of the genome, in-vitro characterization of the transcriptome, in-vitro characterization of the proteome, in-vitro characterization of the glycome, in-vitro characterization of the lipidome, in-vitro characterization of the ligandome, in-vitro characterization of the metabolome, in-vitro characterization of the signalome and combinations thereof.

The in-vitro characterization of tumour tissue of a glioblastoma of a patient may include a determination of whether, in the tumour tissue of the patient, a molecule selected from the group consisting of DNA, mRNA, protein, peptide, sugar, lipid, ligand, metabolite, signal transduction pathway molecule and combinations thereof shows an abnormality, in particular an abnormal molecular structure, the abnormality optionally consisting of a deviation in the sequence, structure, function, concentration and/or activity of DNA, mRNA, protein, peptide, sugar, lipid, ligand, metabolite and/or signal transduction pathway molecule.

The oncological medicament of the combination of medicaments provided preferably comprises temozolomide or consists thereof. Temozolomide has the advantage of low to acceptable toxicity. The dosage may be 40 mg/m² body surface area.

The at least nine particular non-oncological medicaments of the combination of medicaments provided comprise preferably the medicaments aprepitant, minocycline, disulfiram, celecoxib, sertraline, captopril, itraconazole, ritonavir and auranofin or consist thereof.

The list of non-oncological medicaments from which a selection is made in the method according to the invention preferably comprises or consists of one or more medicaments for which an inhibitory effect on an oncogene associated with glioblastoma is known. The non-oncological medicaments in this list preferably have an inhibitory effect on glioblastoma processes selected from the group consisting of growth processes, migration processes, invasion processes, tumorigenic processes, antiapoptotic processes and combinations thereof. Particularly preferably, an inhibitory effect on a growth factor, a growth factor receptor, an oncogene and/or signal transduction pathways of a glioblastoma, which mediate proliferation, invasion, tumorigenicity and/or apoptosis resistance, is known.

In a preferred embodiment of the method, the selection in the method of the at least one further medicament comprises the following steps:
i) comparing the result for the characterized tumour tissue from the patient with the results of a large number of molecular in-vitro characterizations of tumour tissues of glioblastomas in a large number of other patients, the results for the large number of other patients being stored on a data carrier;
ii) determining whether, following the comparison, there are abnormalities present;
iii) matching the abnormalities to at least one non-oncological medicament from the list of non-oncological medicaments, there being a correlation available from the data carrier between abnormalities and the non-oncological medicaments on the list; and
iv) selecting at least one further medicament from the list of non-oncological medicaments on the basis of this match, with in particular those medicaments to which no abnormality could be matched being deleted from the selection and thus not selected.

A “correlation” as mentioned above is present when the detected abnormalities (i.e. the analytically documented molecular aberrations in the tumour tissue) represent target structures of the non-oncological medicaments concerned. Whether this is the case, i.e. whether the target structures can be matched to particular non-oncological medicaments, can be ascertained from the data carrier (e.g. from a database). In other words, the match can be established from the data carrier (e.g. with the aid of a software algorithm) or is (already) stored on it.

The abovementioned steps i) to iv) are preferably software-assisted, more preferably via at least one algorithm, most preferably via at least one algorithm stored on a data carrier, with the algorithm in particular executed by a computer.

The data carrier used may be updatable or be updated during the execution of the method, preferably in line with the current state of knowledge, in particular based on findings from scientific publications and/or studies.

In a preferred embodiment of the method, a selection is made in step c) of at least 2, preferably at least 5, more preferably at least 10, in particular at least 15, medicaments in the list of non-oncological medicaments.

In a further preferred embodiment of the method, the list of non-oncological medicaments used in step c) comprises at least 150, preferably at least 200, non-oncological medicaments or consists thereof.

The invention further provides a data carrier on which software is stored. The data carrier is characterized in that the software stored on it is programmed to execute the following steps:
i) comparing a result of the molecular in-vitro characterization of tumour tissue of a glioblastoma of a patient with results of a large number of molecular in-vitro characterizations of tumour tissues of glioblastomas in a large number of other patients, the results for the large number of other patients being stored on a data carrier;
ii) determining whether, following the comparison, there are abnormalities present;
iii) matching the abnormalities to at least one non-oncological medicament from a list of at least 100 non-oncological medicaments, there being a correlation available from the data carrier between abnormalities and the non-oncological medicaments on the list; and
iv) selecting at least one further medicament from the list of non-oncological medicaments on the basis of this match, with in particular those medicaments to which no abnormality could be matched being deleted from the selection and thus are not selected.

A “correlation” as mentioned above is present when the detected abnormalities (i.e. the analytically documented molecular aberrations in the tumour tissue) represent target structures of the non-oncological medicaments concerned. Whether this is the case, i.e. whether the target structures can be matched to particular non-oncological medicaments, can be ascertained from the data carrier (e.g. from a database). In other words, the match can be established from the data carrier (e.g. with the aid of a software algorithm) or is (already) stored on it.

The software stored on the data carrier allows the results of molecular in-vitro characterizations of tumour tissues of a glioblastoma in a particular patient undergoing treatment to be swiftly compared with results of a large number of molecular in-vitro characterizations of tumour tissues of glioblastomas in a large number of other patients. In addition, the software permits the swift matching of abnormalities in the results to at least one non-oncological medicament from a list of at least 100 non-oncological medicaments. The software thus permits swift identification of at least one promising medicament from the list, i.e. swift drug-target matching.

The data carrier can be characterized in that it is updatable, preferably updatable in line with the current state of knowledge, in particular updatable on the basis of findings from scientific publications and/or studies. The ability to be updated has the advantage of also allowing the storage of correlations that are discovered at a later date between abnormalities in a characterized tumour tissue and the non-oncological medicament that is of benefit for this. The ability of the data carrier to be updated likewise means that drug-drug interactions that are in future found to be advantageous or disadvantageous may be stored on the data carrier or that the data carrier may be updated accordingly. This process also means, for example, that particular non-oncological medicaments that are found to have a high potential for pharmacological interactions (“offending drugs”) can be deleted from the stored list. For offending drugs with the same potential for pharmacological interactions, a guideline may additionally be drawn up and stored accordingly on the data carrier. In addition, it is possible, on the basis of published scientific evidence, to delete from the list non-oncological medicaments expected to have lower anti-glioblastoma effectiveness in favour of non-oncological medicaments expected to be more effective.

The data carrier may be selected from the group consisting of hard disk, flash memory, CD, DVD, Blu-Ray, server and combinations thereof. Preferably, the data carrier is a server that can be accessed via the internet.

The data carrier can be characterized in that it is contained within a computer and/or is accessible from a computer, the software preferably being executable by the computer.

The data carrier can be further characterized in that the data carrier has stored on it a large number of characterized tumour tissues of glioblastomas in a large number of patients.

The data carrier can be further characterized in that a correlation between abnormalities in an in-vitro characterization of tumour tissues of a glioblastoma of a patient and non-oncological medicaments is stored on the data carrier and/or can be determined from information stored on the data carrier by the software stored on the data carrier. A “correlation” as mentioned above is present when the detected abnormalities (i.e. the analytically documented molecular aberrations in the tumour tissue) represent target structures of the non-oncological medicaments concerned. Whether this is the case, i.e. whether the target structures can be matched to particular non-oncological medicaments, can be ascertained from the data carrier (e.g. from a database). In other words, the match can be established from the data carrier (e.g. with the aid of a software algorithm) or is (already) stored on it.

The non-oncological medicaments are preferably stored in a list on the data carrier that comprises at least 100, more preferably at least 150, in particular at least 200, non-oncological medicaments or consists thereof.

1-15. (canceled)

16. A method for providing a medicament combination, comprising the steps:
a) performing a molecular in-vitro characterization of tumour tissue of a glioblastoma of a patient, with a result of the molecular in-vitro characterization being obtained;
b) providing a combination of medicaments, the combination comprising an oncological medicament for the treatment of glioblastoma and at least nine non-oncological medicaments;
c) selecting at least one further medicament from a list of at least 100 non-oncological medicaments; and
d) combining the combination of medicaments provided in step b) with the at least one medicament identified in step c) into a medicament combination;
wherein the selection of the at least one further medicament in step c) is based on the result of the molecular in-vitro characterization in step a).

17. The method according to claim 16, wherein the molecular in-vitro characterization of tumour tissue of a glioblastoma of a patient is selected from the group consisting of in-vitro characterization of the genome, in-vitro characterization of the transcriptome, in-vitro characterization of the proteome, in-vitro characterization of the glycome, in-vitro characterization of the lipidome, in-vitro characterization of the ligandome, in-vitro characterization of the metabolome, in-vitro characterization of the signalome and combinations thereof.

18. The method according to claim 16, wherein the in-vitro characterization of tumour tissue of a glioblastoma of a patient includes a determination of whether, in the tumour tissue of the patient, a molecule selected from the group consisting of DNA, mRNA, protein, peptide, sugar, lipid, ligand, metabolite, signal transduction pathway molecule and combinations thereof shows an abnormality.

19. The method according to claim 16, wherein the oncological medicament comprises temozolomide.

20. The method according to claim 16, wherein the at least nine particular non-oncological medicaments comprise aprepitant, minocycline, disulfiram, celecoxib, sertraline, captopril, itraconazole, ritonavir and auranofin.

21. The method according to claim 16, wherein the list of non-oncological medicaments comprises medicaments for which an inhibitory effect on an oncogene associated with glioblastoma is known.

22. The method according to claim 16, wherein the selection of the at least one further medicament comprises the following steps:
i) comparing the result for the characterized tumour tissue from the patient with the results of a large number of molecular in-vitro characterizations of tumour tissues of glioblastomas in a large number of other patients, the results for the large number of other patients being stored on a data carrier;
ii) determining whether, following the comparison, there are abnormalities present;
iii) matching the abnormalities to at least one non-oncological medicament from the list of non-oncological medicaments, there being a correlation available from the data carrier between abnormalities and the non-oncological medicaments on the list; and
iv) selecting the at least one further medicament from the list of non-oncological medicaments on the basis of this match.

23. The method according to claim 22, wherein the data carrier is updatable or updated.

24. The method according to claim 23, wherein in step c)
i) a selection is made of at least 2 medicaments from the list of non-oncological medicaments; and/or
ii) the list of non-oncological medicaments comprises at least 150 non-oncological medicaments.

25. A data carrier, on which software is stored, wherein the software is programmed to execute the following steps:
i) comparing a result of the molecular in-vitro characterization of tumour tissue of a glioblastoma of a patient with results of a large number of molecular in-vitro characterizations of tumour tissues of glioblastomas in a large number of other patients, the results for the large number of other patients being stored on a data carrier;
ii) determining whether, following the comparison, there are abnormalities present;
iii) matching the abnormalities to at least one non-oncological medicament from a list of at least 100 non-oncological medicaments, there being a correlation available from the data carrier between abnormalities and the non-oncological medicaments on the list; and
iv) selecting at least one medicament from the list of non-oncological medicaments on the basis of this match.

26. The data carrier according to claim 25, wherein the data carrier is updatable.

27. The data carrier according to claim 26, wherein the data carrier is selected from the group consisting of hard disk, flash memory, CD, DVD, Blu-Ray, server and combinations thereof.

28. The data carrier according to claim 26, wherein the data carrier is contained within a computer and/or is accessible from a computer.

29. The data carrier according to claim 26, wherein the data carrier has stored on it a large number of characterized tumour tissues of glioblastomas in a large number of patients.

30. The data carrier according to claim 26, wherein a correlation between abnormalities in an in-vitro characterization of tumour tissues of a glioblastoma of a patient and non-oncological medicaments
i) is stored on the data carrier; and/or
ii) can be determined from information stored on the data carrier by the software stored on the data carrier.
